(19) United States
(12) Patent Application Publication
(10) Pub. No.: US 2009/0199857 A1
(43) Pub. Date: Aug. 13, 2009

(54) Method and Apparatus for Reminding user to Replace and/or Service Cpap Apparatus and/or Component Thereof

(75) Inventors: Gregory Robert Peake, New South Wales (AU); Klaus Henry Schindhelm, New South Wales (AU); Dieter Heidmann, New South Wales (AU); Philip Rodney Kwok, New South Wales (AU); Paul Anthony Green, New South Wales (AU); Heike Thiele, New South Wales (AU); Christopher John Baxter, New South Wales (AU); Thomas Evan Miller, Churchville, NY (US); Robert Edward Henry, New South Wales (AU)

Correspondence Address: NIXON & VANDERHYE, PC, 901 NORTH GLEBE ROAD, 11TH FLOOR, ARLINGTON, VA, 22203, US

(73) Assignee: RESMED LIMITED, Bella Vista, New South Wales (AU)

(21) Appl. No.: 12/224,509

(22) PCT Filed: Feb. 28, 2007
(86) PCT No.: PCT/AU2007/000237

§ 371 Date: Jan. 14, 2009

Related U.S. Application Data
(60) Provisional application No. 60/777,564, filed on Mar. 1, 2006.
(60) Provisional application No. 60/881,157, filed on Jan. 19, 2007.

(51) Int. Cl. A61M 16/06; G09D 3/00; G09F 9/37; G08B 5/00

(52) U.S. Cl. 128/206.28; 40/107; 40/446; 340/815.4

(57) ABSTRACT

A mask includes a patient interface and a reminder system provided to the patient interface. The reminder may manifest itself in mechanical, electronic, chemical, and/or electro-mechanical forms, and may be provided to various portions of the patient interface, e.g., mask frame, cushion, hoses, headgear, etc. Moreover, the reminder system may be a stand-alone system, or it may be provided to other portions of the CPAP/PPV apparatus.

### CROSS REFERENCE TO RELATED APPLICATIONS

This application claims the benefit of U.S. Provisional Application Nos. 60/881,157, filed Jan. 19, 2007, and 60/777,564, filed Mar. 1, 2006, each of which is incorporated herein by reference in its entirety.

### FIELD OF THE INVENTION

The present invention relates to the field of CPAP (continuous positive airway pressure) or PPV (positive pressure ventilation) therapy for patients suffering from or otherwise diagnosed with Obstructive Sleep Apnea (OSA), chronic obstructive pulmonary disease (COPD) or other breathing related illnesses/diseases. In particular, the invention relates to a method and apparatus to provide a timely reminder as to when to service or replace the CPAP apparatus or component thereof. Preferably, the reminder will be provided directly to the CPAP/PPV patient, although the reminder could also be used as a reminder to a clinic, medical equipment provider/manufacturer, clinician, technician and/or doctor, etc.

### BACKGROUND OF THE INVENTION

CPAP/PPV therapy involves the use of a blower or flow generator to generate a supply of breathable gas that is typically pressurized in the range of about 4-20 cmH₂O. The pressurized gas, in most cases this is ambient air, is delivered to a patient interface using an air delivery conduit. The patient interface can take many forms, including nasal only masks, nasal-oro masks, nozzles, etc., all of which are commercially available from ResMed.

In the course of CPAP/PPV therapy, certain components are subject to various types of use and will naturally require some sort of service, maintenance or repair, and in some cases replacement. For example, the mask cushion (usually made of silicone) is subject to various mechanical stresses, e.g., compression and/or tension forces, because some degree of positive contact is generally beneficial to achieve a seal that is adequate to prevent undue leakage of the pressurized gas and/or to achieve a comfortable, secure fit with the patient. The mask (and cushion) is supported on the user's/patient's head using headgear that can be sufficiently tensioned to create and/or maintain the seal. In addition, contact with or exposure to oils of the user's skin, facial creams, nicotine, high temperatures, sunlight and other uncontrollable activities can cause soiling and/or disrepair of the mask cushion.

Over time, it is generally recommended that the mask cushion be washed, which can be conveniently done manually or in a dishwasher. However, because the sealing and/or other mechanical/tactile properties of silicone may deteriorate with repeated use and/or washing, and/or the silicone may simply have an undesirable visual appearance that may discourage compliance with therapy, many manufacturers recommend that the mask cushion be replaced after six months' usage. In some cases, the entire mask assembly is replaced at the same time because other components of the mask assembly become aged, such as the headgear. The actual time period when the component technically needs to be replaced may vary from patient to patient, depending on whether the mask is properly washed on a regular basis and the patient's specific chemical interaction with the mask cushions, but six months is generally the average. Moreover, for mask cushions, it may be possible to be reimbursed for the cost of the cushion through insurance. Currently, in the United States, insurance will typically reimburse a patient (or health care provider) for a replacement mask cushion every six months, assuming it can be shown that the patient is actively engaged with continuous therapy.

Although it is recommended that replacement of the cushion/mask occur every six months, it has been found that in most cases the patients replace the cushion at much longer intervals, e.g., on an annual basis. Moreover, the failure the properly maintain and/or replace the mask can result in less than optimum treatment, and even injury to the patient, e.g., pressure sores on the patient's cheeks or the nasal bridge area.

In one prior art, low-cost solution from Fisher and Paykel, a reminder in the form of a simple sticker is provided. When the patient commences use of the mask, a sticker with the calculated end-of-life date is affixed to the patient's calendar. The patient uses the mask and is expected to order a new mask when he or she sees the sticker on the calendar. In another prior art reminder system available from Respironics, the mask package is provided with a business card that provides a reminder as to when to replace the mask. That is, the business card includes a list of calendar months and an instruction to circle the month at which the patient becomes eligible for a new mask.

In ResMed's PCT International Publication No. WO 2005/063327, filed 22 December 2004, incorporated herein by reference in its entirety, various mask systems are described that have the characteristic of displaying the approximate “age” of the mask. For example, in one embodiment, stress whitening of the mask provides a visual indication of “age”, e.g., repeated bending of various components causes stress whitening and therefore a signal to the hospital technician to replace the mask. In another embodiment, the aging characteristic can be provided by using materials (e.g., in the headgear and/or frame) that display an accumulation of grime, soiling, etc., due to contact with the patient's skin, sweat, oils, facial secretions, etc. While these aging characteristics may be entirely suitable to signal a clinician or nurse in the context of hospital use, there may be more practical methods for signaling of the need to service or replace the mask in the home use environment. In the home-use environment, patients typically will not readily recognize the deterioration of or changes in the mask because this happens in a slow progression over weeks and/or months.

Therefore, a need has developed in the art to develop a reminder or reminder system for the patient to service and/or replace the CPAP apparatus or a component thereof, such as the mask cushion.

### SUMMARY OF THE INVENTION

One aspect of the invention relates to providing a reminder to a (home-use) patient to service or replace his/her CPAP/PPV apparatus or a component thereof, such as the mask cushion, mask, air delivery conduit, headgear, etc. It is thought that such a reminder will help to enhance therapy and the effectiveness thereof. While the reminder can be used for the patient who carries out therapy in a home setting, the reminder is also suitable for clinical or hospital use.

Another aspect is to encourage patients to use well-maintained equipment to enhance therapy. The encouragement may be in the form of a reminder for the patient to regularly clean the mask cushion, in which case the component will have a longer service life. This can help cut costs to the insurance industry, since the masks will not need to be replaced as often.

According to one embodiment of the present invention, there is provided a mask comprising a patient interface; and a reminder system provided to the patient interface. The patient interface may include, but is not limited to, full face masks, nasal masks, nasal prongs, nozzles or puffs, and related componentry, e.g., headgear, air delivery tubes, etc. The reminder may manifest itself in mechanical, electronic, chemical, and/or electro-mechanical forms, and may be provided to various portions of the patient interface, e.g., mask frame, cushion, hoses, headgear etc. Moreover, the reminder system may be a stand-alone system, or it may be provided to other portions of the CPAP/PPV apparatus.

Other aspects of the invention are directed towards, various methods for providing a reminder, e.g., to a patient and/or a reminder system, e.g., to a mask.

Other aspects, features, and advantages of this invention will become apparent from the following detailed description when taken in conjunction with the accompanying drawings, which are a part of this disclosure and which illustrate, by way of example, principles of this invention.

### BRIEF DESCRIPTION OF THE DRAWINGS

The accompanying drawings facilitate an understanding of the various embodiments of this invention. In such drawings:

FIGS. 1-2 illustrate a reminder system according to a first embodiment of the present invention; FIGS. 2A-2D illustrate process for activating a reminder system according to an embodiment of the invention;

FIGS. 3-4 illustrate a cushion including a reminder system according to another embodiment of the present invention;

FIG. 5 illustrates a schematic diagram of a reminder system including a sensor, according to an embodiment of the present invention;

FIG. 6 illustrates a schematic diagram of an electronic reminder system according to another embodiment of the present invention;

FIG. 7 illustrates a schematic diagram of another electronic reminder system according to an embodiment of the present invention;

FIG. 8 is a side view of the electronic reminder system of FIG. 7;

FIG. 9 is a timing chart explaining operation of a reminder system according to an embodiment of the present invention;

FIG. 10 illustrates a push button according to an embodiment of the present invention;

FIGS. 11-12 illustrate a push button activator element according to an embodiment of the present invention, in first and second positions;

FIGS. 13-14 illustrate a push button activator element according to an embodiment of the present invention, in first and second positions;

FIGS. 15-16 illustrate a “shock” based activator element according to an embodiment of the present invention;

FIGS. 17-18 illustrate a magnetic based activator element according to an embodiment of the present invention;

FIGS. 19-20 illustrate a reminder system according to an embodiment of the present invention;

FIG. 21 illustrates a reminder system according to an embodiment of the present invention;

FIGS. 22-24 illustrate a reminder system according to an embodiment of the present invention;

FIGS. 25-29 illustrate a reminder system according to an embodiment of the present invention; and

FIGS. 30-36 illustrate a reminder system according to an embodiment of the present invention.

### DETAILED DESCRIPTION OF ILLUSTRATED EMBODIMENTS

The typical life of a breathing mask depends on various factors, such as direct exposure to sunlight, number of cycles in the dishwasher, mechanical stress, chemical cleaners, facial skin creams, etc., each of which may adversely affect mask quality and/or mask life. Strongly dependent on the mask quality is the success of the therapy, e.g., a dirty or soiled mask could be the reason a patient begins suffering, e.g., due to ulcerous skin irritation and/or painful sores. Thus, the life of the mask is not unlimited. A good sealing mask that is in good condition will be more comfortable for the patient and generally will result in less trips to the clinician, doctor or sleep lab, which should helpfully reduce costs.

The present invention is directed to the development of a reminder or reminder system in which the patient is provided with a reminder or indicator as to when it is recommended to service, repair, wash, and/or replace a mask. The reminder/system is preferably low cost, washable, easy to use, and/or easy to hear/see. The indicator can be configured such that deterioration of the mask is (visually) signaled to the patient, to prompt cleaning or replacement. Otherwise, the indicator or reminder can simply be indicative of when it is recommended to replace the mask, e.g., an indication of the manufacturer's guaranteed service life of the mask.

While the examples provided herein are directed to the provision of a reminder system for a mask, it should be noted that the reminder or reminder system could be provided for additional therapy components, e.g., the flow generator, filter, humidifier tub, air delivery tube, individual components of the mask, etc. It will be appreciated that each of these components may have different schedules of repair/replacement.

### 1.0 Reminders Based on Chemicals and/or Compositions and/or Changes in Appearance

### 1.1 Tag

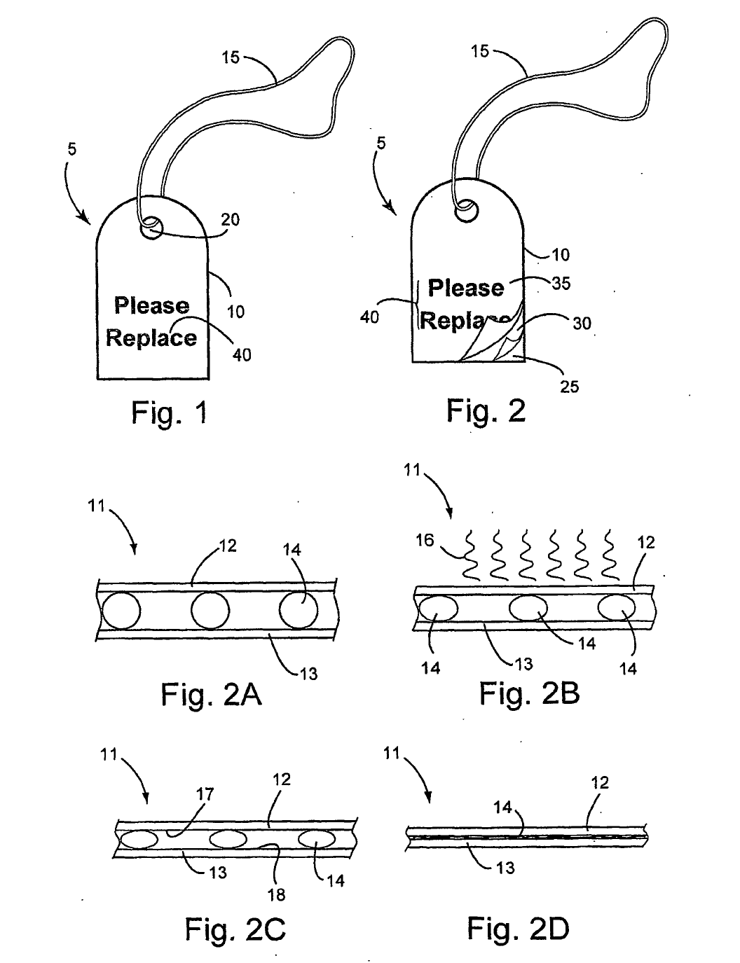

FIGS. 1-2 illustrate a reminder system 5 according to a first embodiment of the invention. In this embodiment, system 5 includes a tag or hanger 10 provided or otherwise connected to a portion of the patient interface, such as the headgear, elbow, etc. In this example, tag 10 includes a lanyard 15 or simply a length of string, etc. that is threaded through a hole 20 and secured to the patient interface.

As shown in FIG. 2, tag 10 includes a layered structure, including a substrate 25, an intermediate layer 30, and a cover 35 (that is preferably transparent). The intermediate layer 30 is preferably encased between the substrate 25 and the cover 35 in a waterproof manner, and includes a chemical or composition that changes with time after activation (activation can occur by simply affixing the cover, substrate and middle layer). For example, the intermediate layer may include a dye that diffuses from the intermediate layer to the cover. For example, after a predetermined period of time, e.g., three months, 4.5 months, six months, etc., the chemical layer (fully or more fully) activates to provide a message 40 to the patient, e.g., in the form of a series of colored stripes, or a text message such as “PLEASE REPLACE” mask.

In an alternative, the chemical or composition of the intermediate layer can be released, activated or otherwise revealed on a temperature dependent basis, for example, using an iron or hairdryer to melt away an isolating layer similar to how touch screen technology works. As shown in FIGS. 2A-2D, tag 10 may include two layers 12, 13 that are separated by small bumps or balls 14. Using an iron or hairdryer to create heat 16 will melt the bumps 14 away and two interior surfaces 17, 18 covered with chemicals come into contact, or the melted state of the bumps 14 simply allows the surfaces 17, 18 to communicate.

The “PLEASE REPLACE” message is not displayed until the predetermined time period has elapsed. Prior to that time, the message is either invisible or only the outline of the message is visible. When the predetermined period of time has elapsed, the chemical ingredient fills in the spaces (or changes its color) such that the message converts from outline appearance to solid appearance.

1.1.1 Multiple Messages and/or Predetermined Times

In addition or in the alternative, tag 10 could provide maintenance messages on a periodic basis (every week or month), e.g., “PLEASE WASH”. If the cleaning message is to be provided in addition to the replacement message, any different timing for such indicators should be taken into account. One solution would be to provide two intermediate layers, each having a chemical or composition that reveals itself after the desired time period has elapsed. Another alternative would be to provide two different chemicals on the same intermediate layer, with each chemical or composition being activated at the desired time period. As an alternative to the messages “PLEASE REPLACE” and “PLEASE WASH”, the use of different colors is contemplated, e.g., yellow to wash and red to replace, etc.

### 1.1.2 Resettable

Washing of the mask/cushion is typically carried out by running the mask/cushion through a dishwasher. It is contemplated that the “PLEASE WASH” message could be deactivated after the washing has occurred. For example, the chemical or composition that produces the message could be neutralized or deactivated if the mask tag 10 is exposed to high temperature air and/or water that is associated with dishwashers, but not normal use. In that event, the patient would be reminded every time the mask needs washing. Moreover, it is contemplated that the “PLEASE REPLACE” message is not resettable, while the “PLEASE WASH” message is resettable.

### 1.1.3 Activate Message After Predetermined Event

The “PLEASE REPLACE” message could also be initiated after a predetermined event, e.g., a predetermined number of cycles through the dishwasher. For example, it is recommended that most masks be washed every week or month. The chemical layer could be activated to indicate “PLEASE REPLACE” if exposed to a predetermined number of dishwasher cycles, e.g., six cycles of the mask is recommended to be cleaned every month and the expected end-of-life is 6 months, or 20-26 cycles if the mask is recommended to be cleaned on a weekly basis.

### 1.1.4 Activate Based on Timer

Of course, the predetermined event could be the elapse of a predetermined amount of time, in which case the chemical or composition could be activated after the elapse of the desired time period, e.g., if used in conjunction with a timer that sends a signal to activate the chemical layer. At least one embodiment using a timer is described below in more detail.

### 1.1.5 Mask Includes Chemical Layer or Composition

While the chemical or composition is described above as being provided on a tag, it is also contemplated that a chemical layer or composition could also be provided on, embedded into, or formed as part of the mask itself e.g., the mask frame, cushion elbow, headgear, swivel connector, etc.

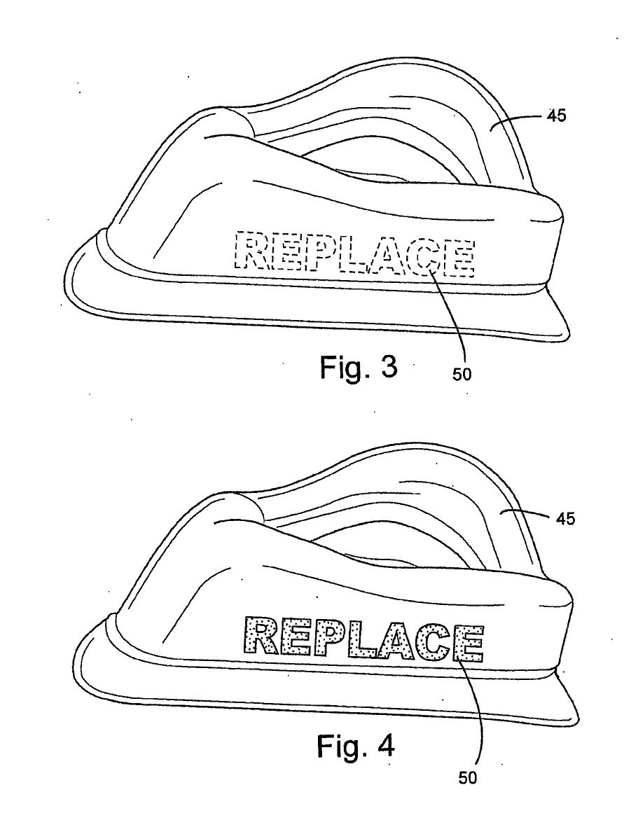

For example, FIGS. 3 and 4 show a cushion 45 having a message 50, e.g., “REPLACE”, that appears after a predetermined amount of time after mask usage occurs. The letters of the message are made up of strips of material that change color, like the tag 10 described above.

### 1.2 Lipid-Phyllic

In another embodiment, the mask cushion can be made of or include a lipid-phyllic (LP) silicone or other material that may or may not show signs of wear over time. The LP silicone can be made entirely of LP silicone, in which case the entire cushion begins to look yellow, a visual signal that should trigger the patient into replacing the mask.

In another alternative, the cushion can be constructed such that is has an encased or embedded layer (within the thickness of the cushion) including one or more cells or pockets that are normally transparent when the cushion is relatively fresh. The cells or pockets can take the form of lettering (“REPLACE CUSHION”) and/or logos, etc., anything that will provide a visual indication to the patient. As the cushion wears and approaches its end of life, the cells or pockets begin to change color, thereby revealing the message. The cells or pockets may include a material that reacts with the patient's fluids (e.g., sweat, saliva, lipids, oils, etc.) so as to result in a change in color of the material in the cells/pockets or the pockets themselves. For example, the material may be an LP silicone that changes colors once the lipids from the patient, over time, penetrate to a predetermined depth where the cells or pockets are located, thus revealing the message, e.g., “REPLACE”. In reference to FIGS. 3 and 4, the pockets or cells of patient-fluid-activatable material take the form of the letters of the “REPLACE” message.

### 1.3 Sensor

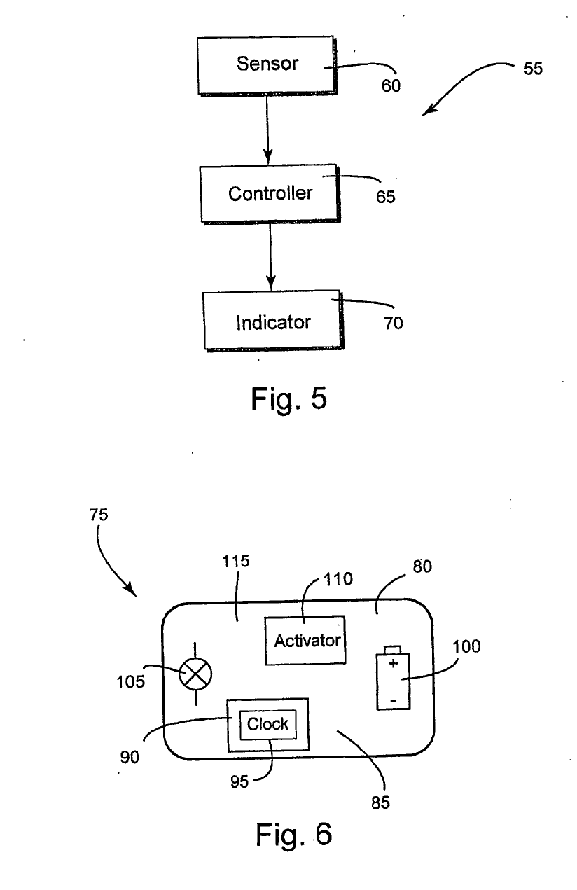

In the above embodiments, a (visual) signal is provided to the patient to indicate the need to wash or replace a mask or cushion. In another embodiment or variant of the present invention shown schematically in FIG. 5, system 55 includes a sensor or monitor 60 to monitor or sense the occurrence of an event indicative of aging of the mask, and to generate a signal (audio/visual) that is provided to a controller 65 that in turn activates an indicator 70 (light, beep, etc.) to alert the patient as to recommended action.

### 1.3.1 Transparency Sensor

Sensor 60 can be provided to detect changes in the appearance of the cushion over time. In use, the cushion changes its appearance, e.g., by turning from relatively transparent to relatively opaque (due to body oils, fluids, creams, etc.). Other components may show age by other measurable changes, etc., stress-whitening (due to repeated bending), etc. Thus, the sensor could sense this condition or transition and provide a signal to the patient.

### 1.3.2 Patient Fluid Sensor

In another variant, the sensor could be embedded within the thickness of the cushion and send a signal when the patient's fluids have penetrated to a predetermined depth. For example, the cushion can be made of a lipid-phyllic silicone that allows penetration of the patient's lipids over time, e.g., 3 months, 6 months, etc., and when such penetration occurs, a signal is sent to the patient to take action (wash, replace, etc.).

### 1.3.3 Conductivity Sensor

In yet another embodiment, a sensor can be used to monitor predetermined properties of the cushion. For example, the presence of lipids or other bodily fluids may change the conductivity (or other properties) of the cushion. If a predetermined amount of lipids are present on or in the cushion, the conductivity monitor generates a signal to the patient to repair/replace the mask.

### 1.4 Hyper-Color Embodiment

In another embodiment, at least a portion of the mask, e.g., cushion, may be constructed of a hyper-color fabric or material that changes color as the patient breathes through it. The color change may indicate that the mask is working properly. The ability to change color may wash out after a number of washes (e.g., 5-100, preferably about 50) prompting the patient to obtain a new mask.

### 2.0 Electronic

FIG. 6 is a schematic drawing of a reminder system 75 according to an embodiment of the present invention. Reminder system 75 includes an electronic system 80 including a substrate 85 that is provided with a controller 90 having a clock/timer 95, power supply 100, indicator 105, and an activator element 110. A shell 115 covers the assembly in a sealed manner.

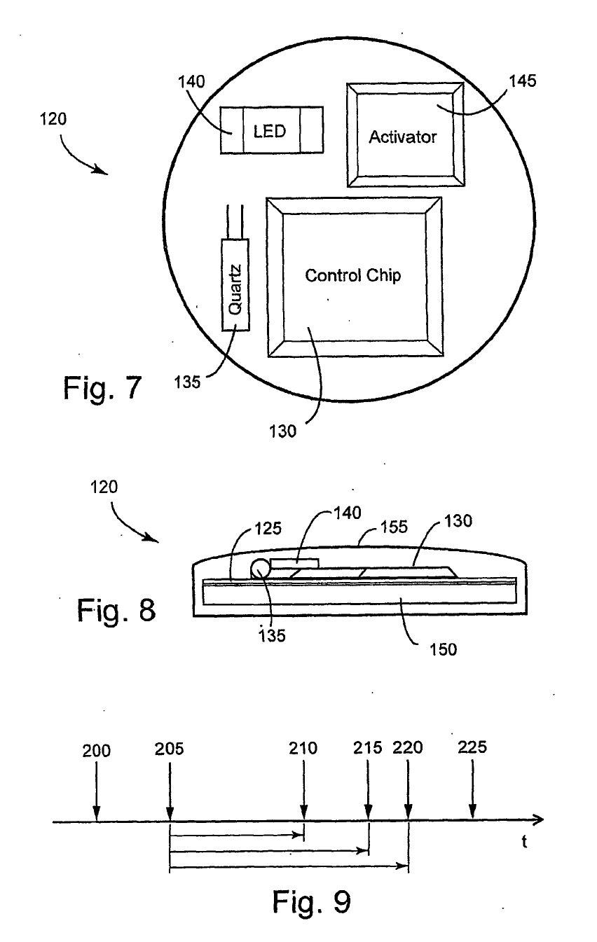

FIG. 7 is a plan view of an electronic reminder system 120 that is designed in the form of a button, and FIG. 8 is a cross-sectional side view thereof. System 120 includes a substrate 125 in the form of a printed circuit board (PCB) that supports a control chip 130 (with a timer/clock), quartz element 135, an indicator 140 (e.g., LED), and an activator element 145. Substrate/PCB 125 is mounted on a power supply 150, e.g., a battery (e.g., a standard or customized cell such as an Li-Ion cell, which is good for use in closed systems), and a shell 155 covers the entire assembly. The internal components of system 120 are preferably completely sealed by the cover, relative to the mask, such that its operation is not adversely affected by normal use and cleaning of the mask, and vice versa.

### 2.1 Timing Of Operations

FIG. 9 is a timing chart that maps out the intended operation of the electronic reminder system. Initially, at point 200, the mask is manufactured. At this time, a function test is performed to ensure that the mask operates correctly. The function test can include a test to determine whether the electronic reminder system is operable. During this test, the device self-tests and indicates the results of the test via the indicator 105, 140.

Between points 200 and 205, the system is off. The time between points 200 and 205 corresponds to that time when the product is being shipped or delivered from the manufacturer/distributor (provider) to the patient.

At point 205, the electronic system 75, 120 is activated, e.g., using the activator element 110, 145. Some sort of feedback (flashing of the indicator, beeping etc.) is provided to verify that activation has occurred. Point 205 corresponds to the time the product is issued to the patient and the patient or clinician activates the activator element. While the timer is activated and running, the indicator light is not illuminated (other than as an initial verification that activation has occurred). Thus, the system operates in a so-called sleep or quiet mode during which time is being measured by the timer/clock.

The timer/clock runs until a predetermined amount of time has elapsed. For example, the predetermined time period may be short or long, depending on the component and the action to be taken. Points 210, 215 and 220 are examples or representative predetermined time periods. After the predetermined amount of time has elapsed, the indicator is then activated so as to provide a visual indicator (light) to the patient to take action in regard to the mask. The indicator could also take the form of an audio signal (e.g., a beep).

The indicator is activated until point 225, after which the indicator is deactivated, e.g., due to expiration of the battery. Alternatively, the indicator can be activated for a predetermined time, e.g., a few days or hours, after which it is automatically deactivated so as to reduce possible annoyance to the patient. In this alternative, the indicator can be controlled to again be activated (e.g., after a day or two or a week) in a last effort to urge the patient to replace the mask. In yet another alternative, the system includes a shut off switch or button that can be accessed by the patient if it is desired to immediately deactivate the timer and/or indicator light.

### 2.2 Multiple Modes of Indication

The indicator can be controlled in a staged manner (multiple mode) such that the patient stimulation pattern and/or intensity varies over time (light and/or sound). For example, the indicator may slowly flash (or beep) after the predetermined time period has elapsed (first mode or stage), and then progressively increase its rate of flashing or beeping (second mode or stage), e.g., over a period of a few days.

The indicator can be controlled so as to slowly flash or beep a few days or a week before the predetermined time period has elapsed, and then the indicator light can progress to a more rapid rate of flashing or beeping (or move to a continuous light/sound mode) when the predetermined time has elapsed. This will allow the patient to order the mask just before it reaches its end of life. Also, this staged warning should provide some additional comfort to the patient since the mask can still be used during the early stage. By contrast, a mask that only includes one warning level, e.g., “replace mask”, may cause concern or panic in the patient since it may be unclear whether the mask still has some useful life, and the patient may not want to use a mask which where the end of life indicator has timed out. Staged warning can help avoid the undesirable situation where the patient simply does not use the mask even though the replacement has not yet arrived.

The electronic reminder system is preferably integrated directly into the patient interface, e.g., the frame of the mask assembly. The system is preferably sealed in a closed housing without openings, e.g., so that the system is impervious to washing, saliva, etc.

### 2.3 Stand-Alone Reminder System

In an embodiment, the system can be sold as a stand-alone system that can be attached to any mask, e.g., hook and loop fastener to the headgear, a lanyard, etc. Therefore, the patient could transfer the system from the old, spent mask to the new replacement mask. Further, the system may include a reset button that can be activated by the patient when treatment with the new mask commences.

### 2.4 Programmable

The system could also be programmable, such that multiple reminders for one or more components for action to be taken can be indicated. See, e.g., ResMed's U.S. Provisional application no. 60/626,178, filed Oct. 14, 2005, and U.S. non-provisional application Ser. No. 10/533,940, filed Jun. 21, 2004, each incorporated herein by reference in its entirety. Each discloses various reminders that are integrated into the flow generator, e.g., a menu driven reminder system.

### 2.5 Activator Elements

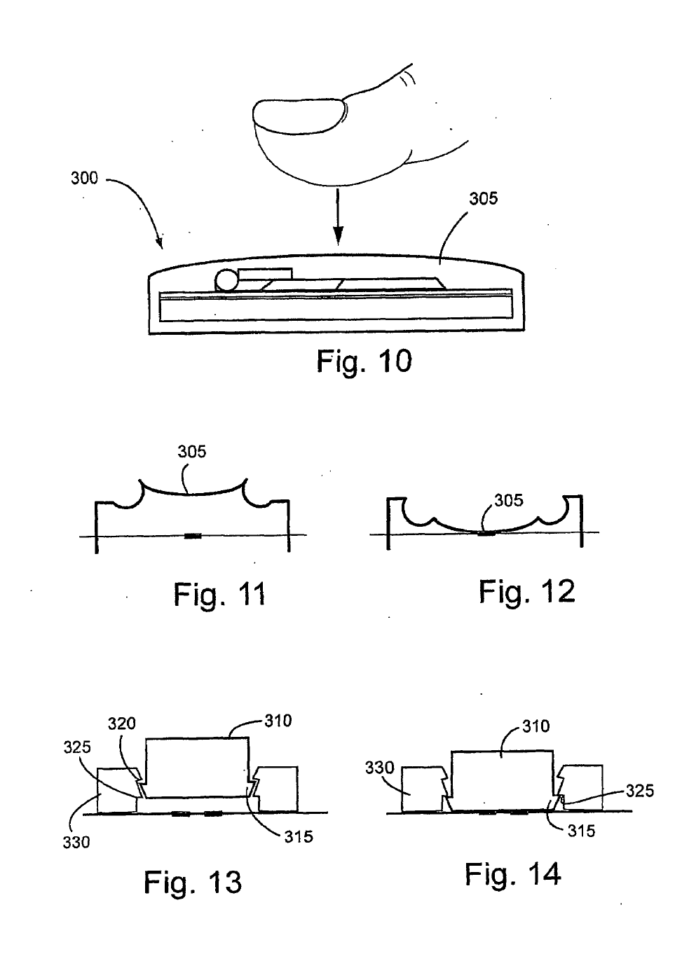

FIGS. 10-18 illustrate electronic reminder systems, each including an activator element. The activator element may take several forms, e.g., mechanical, magnetic, RF, automatic, laser, etc, some of which can be activated by both the patient and the manufacturer/distributor (provider), and some of which can only be activated by the provider. The activator element is preferably activatable only a single time, i.e., one time use.

### 2.5.1 Pushbutton Activator Element

In FIGS. 10-12, the system includes an activator element in the form of a push button 300 that can be pressed so as to trigger the clock/timer. Push button 300 has a housing 305 with a compliant or soft top that is plastically deformable in this example, such that once pushed in, it is permanently deformed in this position such that is activated only once. This structure acts as a switch that can be switched a single time from “off” to “on”. The housing can be made, e.g., of metal or plastic foil, metal or coated plastic, etc. FIG. 11 shows the pre-activation position, while FIG. 12 shows the activation and post-activation position.

FIGS. 13-14 show another button-type embodiment in which a housing 310 can be depressed in button like fashion to activate the timer/clock. Housing 310 includes a flanged member 315 that is engagable with one of two corresponding recessed portions 320, 325 on a stationary part 330, which may be formed as an integral portion of the mask shell/frame or elbow. Housing 310 is depressed from the position in FIG. 13 (the “OFF” position) in which the housing flange engages the upper recess 320, to the position in FIG. 14 (the “ON” position) in which the housing flange engages the lower recess 325 and thereby activates the timer/clock.

### 2.5.2 Shock-based Activator Element

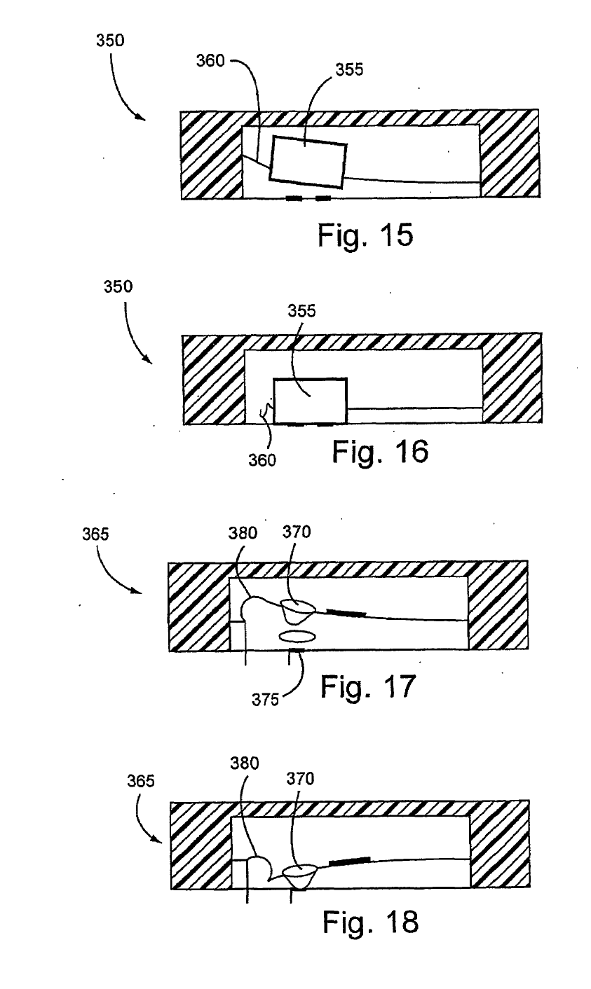

FIGS. 15-16 illustrate an activator element 350 which is operable to activate the timer based on the shock principle. The system 350 has a micro-mechanical construction and is affixed or otherwise provided to the mask, e.g., via adhesive, etc. Shock to the mask will accelerate a small weight 355 in the activator element to thereby break a fixture 360 (e.g., a frangible material such as plastic), which in turn activates the timer/clock. The shock can be applied by simply pressing on the housing until the fixture breaks, or the mask can be shocked by “smashing” or otherwise rigorously handling the mask. Preferably the indicator will provide a sign that the timer has been successfully activated.

### 2.5.3 Magnetic Activator Element

FIGS. 17-18 illustrate an activator element 365 which is operable to magnetically activate the timer. The system also has a micromechanical construction and is affixed to or otherwise provided to the mask, e.g., via adhesive, etc. System includes a magnet 370. To activate the timer, a strong magnet 375 is placed near the back side of the housing to attract magnet 370. This will cause movement of a fixture 380 that activates the timer. Depending on preference and application (e.g., the spring force, dimensions of the arm, the magnet 370 can either break the fixture or simply cause it to move (e.g., stretch) enough to activate the timer, in which case the switch can be suitable for multiple usage.

### 2.5.4 RF Activation Element

The activation element may include radio frequency as a means to activate the timer. This would be similar to a radio frequency identification (RF ID) tag. In this example, the “tag” would be provided to the mask frame/shell/housing, and a strong electromagnetic field would be used to activate the timer via the tag.

### 2.5.5 Automatic Activation of the Self-Test

Activation of the timer can be automatically implemented during the manufacturing process by, for example, connecting the battery. For example, during the self-test, the result is signaled by the indicator after a predetermined time. Then the system shuts off by itself and activation is initiated by the user.

### 2.5.6 Laser Activation

In another embodiment, activation (and even deactivation) of the timer can be accomplished using an activation element in the form of a laser. In this embodiment, the housing of the system is, for example, made of material that can be penetrated by infrared light to activate the timer.

### 2.5.7 Pull Tab

In another embodiment, the activator element can take the form of a pull tab of the type that is available on some children's battery operated toys. The pull tab is preferably made of an electrical insulator material and has a first end that is exposed or otherwise accessible to the patient, and a second end that is interposed between one terminal of the battery and the electrical connector or at some other position that effectively opens the circuit. Removing the pull tag (e.g., by pulling the first end at the time use commences) starts the clock/timer. After a predetermined amount of time has elapsed on the clock/timer, a signal is sent to the patient to take appropriate action.

### 2.6 Battery Indicator Embodiment

In another embodiment, a battery operated indicator may be provided to the mask that is structured to prompt the patient to obtain a new mask. For example, a battery operated message may be activated after a predetermined period of time has elapsed, e.g., message indicating “REPLACE MASK” may light up after a predetermined period of time has elapsed.

In yet another embodiment, an opaque window may be uncovered after a predetermined period of time has elapsed to allow a “REPLACE MASK” message to appear. Specifically, the opaque window may be battery powered to occlude the “REPLACE MASK” message. As battery power is consumed, the occluded message will become transparent or visible.

In still another embodiment, the time for mask replacement may be indicated by a progressive gauge similar to that provided on batteries that indicate their life. For example, the progressive gauge may be structured such as those described in the article at www.washingtonpost.com/wp-srv/photo/essays/vanRiper/030110.htm and U.S. Pat. Nos. 5,557,208 and 5,596,278, each of which is incorporated herein by reference in its entirety. In use, a battery may be operated to consume power over a predetermined period of time that is associated with the time for mask replacement. A progressive gauge may be provided to the battery so that the patient can easily determine when the time for mask replacement is forthcoming.

A descriptor flag or message, e.g., “REPLACE MASK” message, may be a preferred mode of communication over a flashing lamp, for example, as the flashing lamp may provide a potential source of irritation.

### 3.0 Mechanical

### 3.1 Wheel

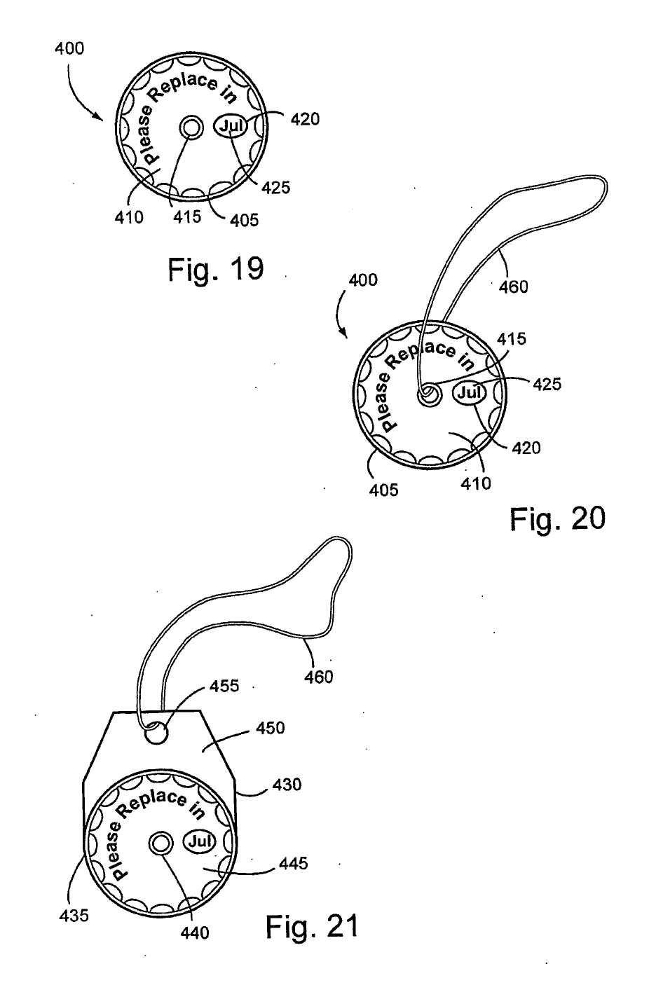

FIGS. 19-20 illustrate a reminder system 400 according to yet another embodiment of the present invention. System 400 includes a substrate in the form of a back wheel 405 and a front wheel 410 that is coupled to the back wheel, e.g., via a sliding axle or duffer 415 that allows the front and back wheels to rotate relative to one another.

Upon relative rotation or other relative movement, a hole 420 in the front wheel aligns with various messages 425 that are printed on a surface of the back wheel. In this example, the messages are the 12 months of the calendar year, so that the patient or provider can align the hole 420 with the correct month (e.g., six months from the date the patient begins to use the mask).

As shown in FIG. 21, back wheel 405 may be in the form of a plate 430 that includes a circular section 435 where the sliding axle or duffer 440 is centered, to couple the front wheel 445 to the plate. The plate 430 may include a supplemental portion 450 that may have space for additional messages or advertising. In addition, the supplemental portion may include a hole 455 through which a string or lanyard 460 can be threaded, to thereby fasten or secure the system to the mask. Moreover, the axle 415, 440 may include a through hole for this purpose (see FIG. 20).

3.1.1 Direct Molding with Mask

The bottom plate or wheel can also be integrated directly onto the mask. For example, the bottom plate or wheel, with the message (months of the year) can be molded directly onto the frame of the mask. The front wheel can then be snap fit directly onto the frame. An axle can be directly molded with the frame, or the front wheel can include the axle. Moreover, the bottom plate or wheel could be designed in S8 top view shape.

### 3.2 Folding Clip

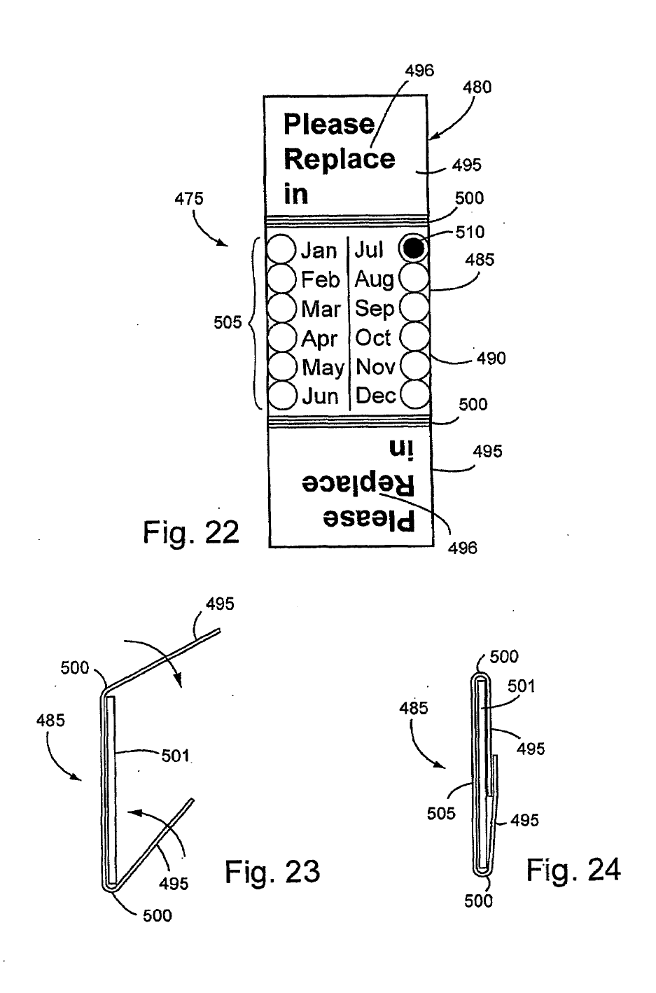

FIGS. 22-24 illustrate a reminder system 475 according to another embodiment of the present invention. System 475 includes a substrate 480 in the form of a folding clip having a central section 490 and flaps 495 that are connected to the central section 490 via predefined bend zones 520. The clip is preferably made of aluminum that holds its shape when formed, but it can be made of paper or other materials and its flaps simply glued or otherwise affixed to one another or the headgear. Each flap 495 may include a message.496, e.g., “Please replace in . . . ”.

In another variant, flaps 495 could include hook and loop fasteners to secure the system on a headgear strap 501 (FIGS. 23 and 24). Otherwise, the flaps could be eliminated and the rear surface of the central portion could include a hook or loop portion that could be affixed to an existing hook or loop portion already provided as part of the headgear strap.

Central section 490 includes a calendar section 505 with a list of each of the calendar months. The provider or patient marks or hole punches the desired month upon commencing use of the mask, thereby establishing when the mask should be replaced. A pen or the like can be used to punch the paper “dot” corresponding to the selected month, or a pen can be used to simply mark the appropriate circle 510, in this case, “July”.

As shown in FIG. 23, the folding clip 485 is attached to headgear strap 501 by folding the flaps 495 in accordance with the predefined bend zones 520. FIG. 24 shows the final assembly, in which the calendar section is preferably positioned on the exposed side of the strap.

### 3.3 Slide Clip

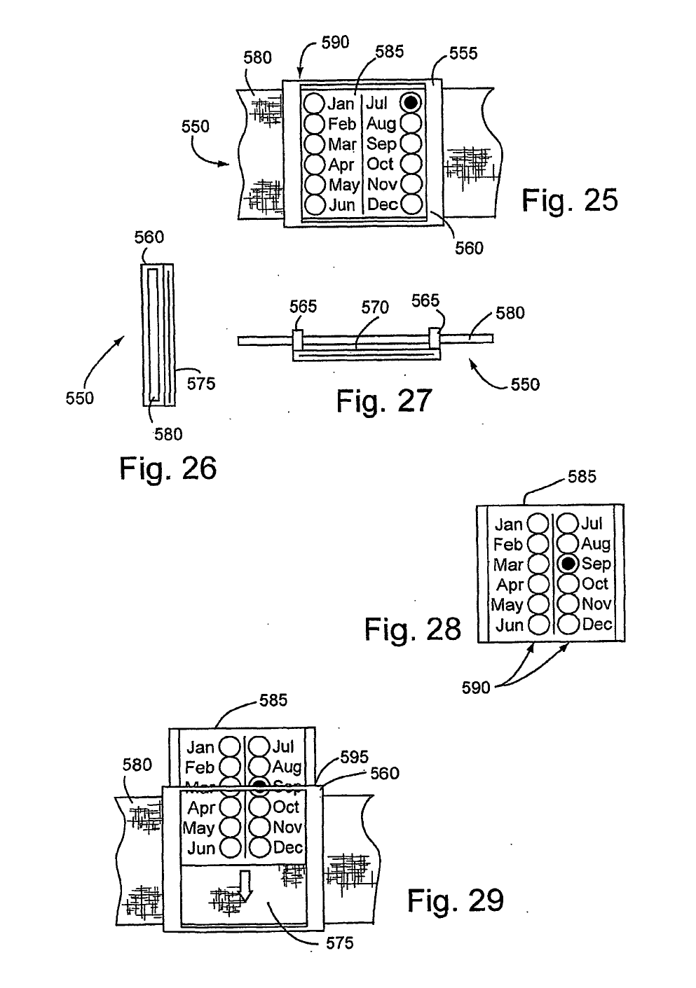

FIGS. 25-29 show a reminder system 550 according to another embodiment of the present invention. System 550 includes a substrate in the form of a clip 555 including main body 560, one or more eyes 565 extending from a first side 570, and a channel 575. Main body 510 is preferably made of molded plastic. The eyes 565 are structured and dimensioned to match the width of and threadedly receive a headgear strap 580. The channel 575 is structured to receive a calendar section 585 having the months of the year printed thereon, along with a column of circles 590 that are marked or punched.

Channel 575 has three closed sides, and one open side 595 provided for purpose of sliding the calendar 585 into the channel. The open side is preferably formed on the top of the strap, although it could be positioned on the bottom or one of the lateral sides. The open side is optionally provided with structure, e.g., a nibble or indentation, to prevent the calendar from inadvertent removal.

### 3.4 Mask Ring Reminder System

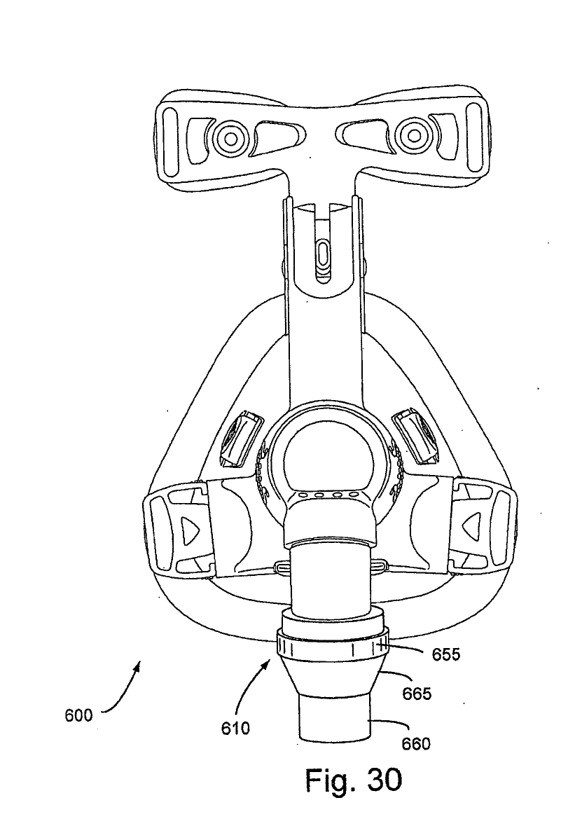

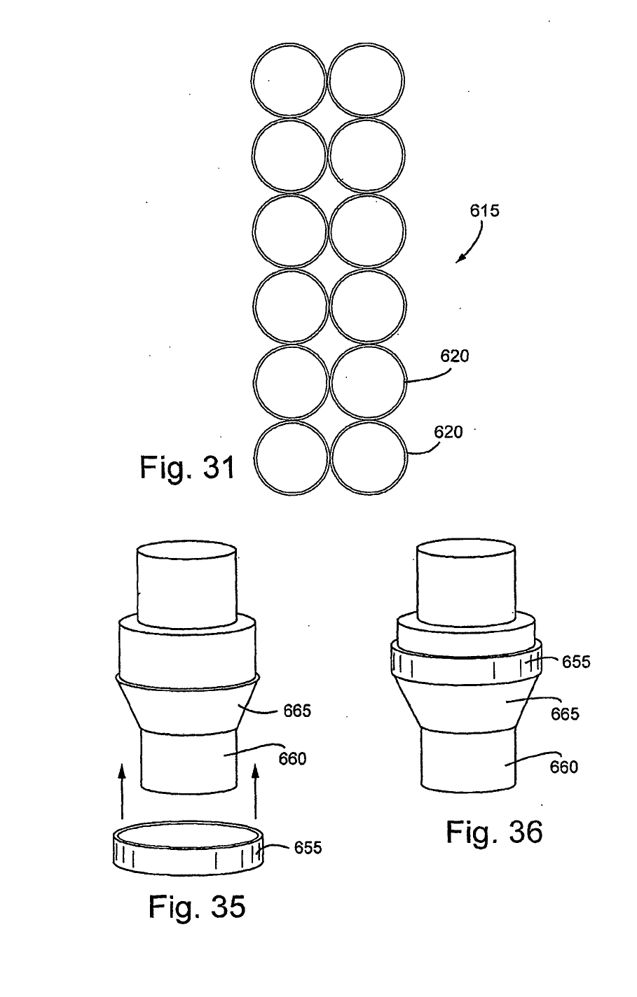

FIGS. 30-36 illustrate a mask assembly 600 including a reminder system 610 according to yet another embodiment of the present invention. System 610 includes a plurality of rings 615 (FIG. 31) that are included within the package in which a mask is sold. In this example, twelve rings 620 are included in the box, each ring corresponding to one of the months of the year. Of course, it is also possible to include only six rings with every other month, e.g., January, March, May, etc., if the reminders are only necessary in two month increments. The rings can be molded as a single unit, with frangible connections that can be easily broken, or the rings can all be separate from one another.

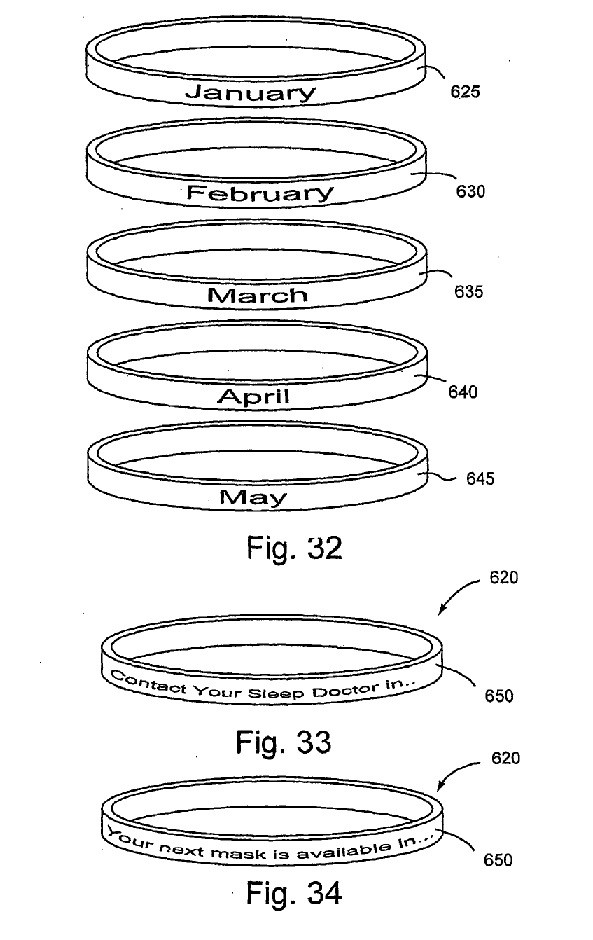

FIG. 32 shows rings 625, 630, 635, 640, 645 for the months of January to May, while FIGS. 33 and 34 show two examples of the type of messages that may be provided to the rear side 650 of the ring 620. In addition, the back side of the ring could provide details, a phone number or an email address of the patient's clinician or mask provider.

Once the ring with the appropriate reminder month is selected, the ring 655 (FIG. 35) is attached to the mask. In this example, the ring 655 is dimensioned to fit over the swivel elbow 660 (or a cylindrical connector thereof) that is provided to direct pressurized gas to the patient. Elbow 660 includes a wedge shaped flange member 665 that retains the selected ring 655 in place. This flange member acts as a “one-way” feature since in practice is desirable that the ring cannot be removed. FIG. 36 shows the ring 655 in its assembled position.

In the above embodiment, the ring is made of a plastic material that is relatively rigid, yet allows a very small degree of stretching to overcome the wedge shaped flange 665.

### 3.4.1 Elbow/Connector With Circumferential Rib

In another embodiment, the cylindrical connector of the elbow can be provided with a circumferential rib that is configured to cooperate with a ring that can stretch over the rib. In this case, the ring is preferably made of an elastic material. The rib may include a groove that cooperates with the rib.

### 3.4.2 Single Ring

In another embodiment, only a single ring is provided and the intended replacement month is hand written directly on the mask by the patient or provider. Otherwise, a sheet of stickers will all the months preprinted thereon can be provided and the patient or provider can select the appropriate sticker for placement on the ring.

### 3.4.3 Months Provided on Elbow/Swivel

In another embodiment, the elbow or the swivel can be printed or otherwise provided with months of the year, and the mask can be sold with a single ring having a hole or slot that can be aligned with the desired year.

### 4.0 Comfort Feature

The mask can include one or more features that are not necessary for operation of the mask, but nonetheless make it more comfortable for the patient. One aspect of the invention is to use these comfort features, or the lack thereof, to prompt the patient into repairing or replacing the mask.

### 4.1 Aromatic

In another embodiment, a portion of the mask system can be made from one or more materials having an aroma, scent, or odor that is detectable by the patient. For example, the mask cushion can include a pleasant scent that helps ease the administration of CPAP treatment.

The scented material can be provided to the mask assembly in a number of ways. For example, the cushion can be dipped in a scented material, or the silicone that is used to make the mask cushion can be mixed in during the manufacturing process. The scented material can be heat-activated such that the scent is more actively released during therapy when the patient's body warmth is applied. In addition, interaction with humidity and/or the patient's bodily fluids (saliva, oil, sweat, etc.) can enhance activation.

In another alternative, the scented material can be provided on a substrate that is attachable to the mask. For example, the substrate may include a hook and loop fastening system that can be selectively attached to an detached from the headgear.

In yet another alternative, the scented material can be applied, e.g., simply sprayed, on one or more components of the mask, e.g., the headgear, cushion, etc. Alternatively, the mask component can be dip-coated with the scented material.

Regardless of which form is used, the scented material can be used as a “feedback” signal to the patient to repair or replace the mask or component thereof. For example, the scented material can be set to have a useful life approximating that of the mask component, e.g., both the scented material and the mask cushion need to be replaced after a predetermined period of time, e.g., 6 months. While the patient may not recognize the need for the mask cushion to be replaced, the lack of the pleasant scent can trigger the patient into ordering a new mask.

### 4.1.1 Repeated Activation

In another manifestation, the scented material can be repeatedly activated. For example, the scented material could be configured to last only a week, at which point the patient would need to wash the mask to again activate the scented material. The water and/or high temperatures associated with the dishwasher could “re-fuel” or re-energize the scented material such that it releases the desired scent for another cycle.

In yet another version, the mask system could be configured to release an (unpleasant) odor to trigger the patient into replacing, repairing or otherwise maintaining the mask.

### 4.2 Massage Mask

In another embodiment, the comfort feature takes the form of a massage mask, such as disclosed in ResMed's U.S. Provisional application No. 60/750,802, filed Dec. 16, 2005, incorporated herein by reference in its entirety.

In this embodiment, the massager is enabled and/or powered by a battery having a battery life that is matched to the anticipated life for the mask, e.g., six months. When the battery wears out, the massage feature is no longer enabled, and the patient will be prompted into replacing the mask.

### 5.0 Other Embodiments

### 5.1 Database Counting

In this embodiment, a mask database can be signaled to activate the clock/timer upon the occurrence of an event. In one example, the event could be the opening of a new mask system that is to be (soon) used by the patient. For example, a card might be provided in the package that the patient mails to their mask distributor who then can activate the database timer. Alternatively, the package or an element therein might prompt the patient to email their mask distributor who then activates the database timer. After the predetermined period of time has elapsed, a signal is generated for the patient to repair/replace the mask.

The signal can take the form of an audio and/or visual signal. In addition, the signal can be an email or SMS message that is automatically sent to the patient and/or provider.

5.2 Connector recognition

In another variant, the mask may be sold with a connector element that, when connected to the flow generator, produces a signal to commence counting. See, e.g., U.S. Provisional application No. 60/656,880, filed Mar. 1, 2005, incorporated herein by reference.

### 5.3 Click Counter

In still another variant, a “click counter” can be provided to the mask and simply count the number of times certain components are clicked into and out of position. For example, the mask typically will include one or more components that click into place, e.g., headgear clips or elbows that click into the frame (e.g., ResMed's Activa TM mask), adjustable yoked headgear arrangements that may click upon adjustment (e.g., ResMed's Vista TM mask), or adjustable forehead supports that may click during adjustment.

One indicator of the age of the mask can be how many times these various clicks or adjustments have taken place. Thus, in one embodiment, a click counter is provided to sense each click or adjustment, and a signal is provided to the patient when a predetermined number of adjustments/clicks has been sensed. Once signaled, the patient can take appropriate action.

### 5.4 Washable Sticker

In another embodiment, the reminder can take the form of a washable sticker. In this embodiment, a sheet of stickers with each of the months is included within the mask packaging. The appropriate month sticker is selected and applied directly to the mask, e.g., on the frame, elbow, etc. The sticker should be relatively rugged such that it can withstand normal use and dishwasher cleansing. In addition, the sticker can have a special shape that is such that it will only correctly fit in a single position on a desired component.

### 5.5 Stand-Alone Reminder System

In still another embodiment, the reminder system can be in the form of a stand alone system that can be separately sold or sold with the mask or other system. For example, the reminder can be in the form of an end of life indicator that commences counting when mask use begins, and provides an alarm (audio/visual) to the patient to take specific action. In one manifestation, the alarm can be a simple timer (e.g., six months) that is mounted on a magnetic substrate or base. As such, the reminder system can be magnetically attached to the patient's refrigerator, or some other metallic surface where the patient is likely to see it.

For example, the magnetic reminder system may be similar to that illustrated at www.balgownievet.com.au/images/ezi_merchant_images/flea_tick_products/frontline/merial_magnet.jpg, which is incorporated herein by reference in its entirety. In such a reminder system, a digital timer is provided that is resetable, e.g., to a predetermined time (e.g., in days) for mask replacement, when the patient pushes a button, e.g., such as a heart-shaped button in the above-noted web image. Once reset, the digital timer counts down (e.g., one each day) until the timer reaches zero. When the reminder system shows zero, the patient understands that it is time to replace the mask or cushion and/or wash, for example. Once the mask or cushion is replaced and/or washed, the timer may be reset back to the predetermined time for mask replacement. In an embodiment, the reminder system may recommend mask or cushion replacement and/or washing at any suitable time period, e.g., once a week to once every five years, preferably once every 6-12 months.

### 6.0 Multiple Metrics of End-of-Life—Sliding Scale

In another embodiment, the predetermined time period can be adjusted based on the sensing of various input. For example, the nominal estimate for replacement of a mask, as suggested by insurance reimbursement schedules and manufacturer's suggestions, is six months. Accordingly, the reminder systems herein are designed such that the patient is signaled after six months that reimbursement for a new mask is available.

However, the mask may have a life that is actually greater or longer than six months, depending on whether the mask is properly maintained. Therefore, one embodiment of the invention includes a system that adjusts the nominal period for replacement of the mask.

For example, there are patient oriented factors and use related factors than can affect the life of the mask. Patient related factors include whether the patient is a smoker, wears creams, sweats profusely, the acidity of their skin, etc. Use related factors may include how many adjustments are made to the headgear straps or forehead support, the number of sensed clicks, the therapy pressure, etc.

The patient and/or use related factors may be taken into account using a questionnaire that is filled in by the patient. Depending on the results, the nominal end of life may be adjusted to accommodate for the specifics of the patient and/or usage. For example, if the patient is a smoker and the therapy pressure is relatively high, the nominal end of life is reduced, e.g., from 6 months to 5 months or less. Of course, while one metric of usage may be negative, another may be positive, in which case the end of life may not be adjusted. Moreover, various metrics may have more impact than others. In this case, the provider can devise a formula by which the nominal life can be adjusted by appropriately weighting the patient factors and the use factors.

In addition, or in the alternative, the use of related factors may be sensed while the mask is used by the patient, e.g., using one or more sensors. In one example, the temperature of the patient is monitored. If the patient's temperature is very high or low, that can be taken into account when re-calculating the end of life, as temperature can affect mask life.

In another example, the number of times the mask is washed is monitored. Washing of the mask can have an effect on the life of the mask. If the mask is not washed on a regular basis, the patient's therapy can be adversely affected, and mask life can be reduced. Therefore, the nominal end of life is reduced, e.g., to 4-5 months, so that the patient is prompted to replace the mask. Obviously, other factors can further reduce the time period, so the formula for adjusting the nominal life should probably include a predetermined minimum life, e.g., 2 months, even if the negative factors might otherwise reduce the nominal life below that amount.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention. Also, the various embodiments described above may be implemented in conjunction with other embodiments, e.g., aspects of one embodiment may be combined with aspects of another embodiment to realize yet other embodiments. In addition, while the invention has particular application to patients who suffer from OSA, it is to be appreciated that patients who suffer from other illnesses (e.g., congestive heart failure, diabetes, morbid obesity, stroke, barriatric surgery, etc.) can derive benefit from the above teachings. Moreover, the above teachings have applicability with patients and non-patients alike in non-medical applications.

1. A mask comprising:
a patient interface; and
a reminder system provided to the patient interface.

2. The mask according to claim 1, wherein the reminder system includes an electronic assembly that includes a controller in communication with a clock/timer, a power supply, an indicator and an activator element.

3-25. (canceled)

26. The mask according to claim 1, wherein the reminder system includes at least one ring indicating the intended month of mask replacement, said ring being provided to a portion of the patient interface.

27. The mask according to claim 26, wherein the patient interface includes a frame and an elbow provided to the mask frame, wherein the ring is provided to the elbow or a connector element associated therewith.

28. The mask according to claim 27, wherein the elbow or element includes a wedge shaped flange, and the ring is structured to be fixed in place by sliding it over the wedge shaped flange until the ring is locked in position.

29. The mask according to claim 27, wherein the ring cannot be removed from the elbow once attached.

30. The mask according to claim 27, wherein the elbow or element includes a circumferential rib that is configured to engage with the ring.

31. The mask according to claim 30, wherein the ring includes a groove to engage the rib.

32. The mask according to claim 1, wherein the reminder system includes a plurality of rings each one having a month of the year appearing thereon.

33. The mask according to claim 1, wherein the reminder system includes a folding clip having a central portion with calendar section including calendar months and at least one flap portion provided to the central portion, the folding clip including a bending zone between the flap portion and the central portion, the at least one flap portion being configured to be folded around a headgear strap to retain the reminder system to the headgear.

34. The mask according to claim 33, further comprising a pair of flap portions each bendable around the central portion.

35. The mask according to claim 34, wherein the flap portions include a hook and loop fastener.

36. The mask according to claim 33, wherein the calendar section includes a plurality of indicators that can be marked or hole punched to indicate the desired month or mask replacement.

37. The mask according to claim 33, wherein the folding clip is made of aluminum configured to maintain its shape in the bent position.

38. The mask according to claim 1, wherein the reminder system includes a calendar section listing a plurality of calendar months, one of which can be indicated for replacement of the mask, the calendar portion having a backing with a hook or loop fastener that is engagable with a hook or loop fastener of headgear associated with the patient interface.

39. The mask according to claim 1, wherein the reminder system includes a sliding clip including a main body with a clip through which a headgear strap may be threaded, a channel and a calendar section listing months of the year and insertable into the channel.

40. The mask according to claim 39, wherein the channel includes three closed sides and a single open side though which the calendar is inserted.

41. The mask according to claim 1, wherein the reminder system includes a front wheel rotatably mounted to a back wheel or plate, the front wheel including a hole that can be rotatably aligned with a desired message on the back wheel or plate.

42. The mask according to claim 41, wherein the message is in the form of months of the calendar year.

43. The mask according to claim 41, wherein the front wheel and the back wheel or plate include a common sliding axle.

44-96. (canceled)
